(19) United States
(12) Patent Application Publication
(10) Pub. No.: US 2023/0192747 A1
(43) Pub. Date: Jun. 22, 2023

(54) CONJUGATION OF PROTEINS TO POLYSACCHARIDES USING A PHOSPHATE BRIDGE

(71) Applicant: Wisconsin Alumni Research Foundation, Madison, WI (US)

(72) Inventor: Srinivasan Damodaran, Middleton, WI (US)

(21) Appl. No.: 18/004,271

(22) PCT Filed: Aug. 11, 2021
(86) PCT No.: PCT/US2021/045515

§ 371 Date: Jan. 4, 2023

Related U.S. Application Data
(60) Provisional application No. 63/064,489, filed on Aug. 12, 2020.

(51) Int. Cl. C07H 15/04; C07H 1/00

(52) U.S. Cl. CPC C07H 15/04; C07H 1/00

(57) ABSTRACT

Conjugates of protein and polysaccharide linked by a phosphate bridge. The conjugates are made by reacting a protein with a polysaccharide in the presence of POCl₃.

### FEDERAL FUNDING STATEMENT

This invention was made with government support under 2018-67017-27561 awarded by the USDA/NIFA. The government has certain rights in the invention.

### BACKGROUND

Conjugation of reducing sugars and polysaccharides with proteins to improve their functional properties has been extensively studied. These studies often employ a Maillard reaction, i.e., Schiff base formation between the reducing end of the carbohydrate and the amino groups in proteins, for conjugation. These methods typically involve heating a homogeneously mixed dry powder at 60-80° C. and at 70-80% relative humidity for a few hours to a few days to catalyze the carbonyl-amine reaction. One of the shortcomings of the dry heating method is that the reaction cannot be controlled. It is not possible to limit the reaction to the initial Schiff base formation. The reaction progresses further and produces undesirable advanced Maillard reaction products, which adversely affect the color and flavor of the product. See:

Akhtar, M.; Dickinson, E. Emulsifying properties of whey protein-dextran conjugates at low pH and different salt concentrations. Colloids Surf B: Biointerfaces 2003, 31, 125-132;

Akhtar M.; Dickinson E. Whey protein-maltodextrin conjugates as emulsifying agents: An alternative to gum arabic. Food Hydrocolloids 2007, 21, 607-616;

Aoki, T.; Kitahata, K.; Fukumoto, T.; Sugiomoto, Y.; Ibrahim, H. R.; Kimura, T.; Kato, Y.; T.Matsuda. Improvement of functional properties of β-lactoglobulin by conjugation with glucose-6-phosphate through the Maillard reaction. Food Res. International 1997, 30, 401-406;

Dickinson, E.; Semenova, M. G. Emulsifying properties of covalent protein-dextran hybrids. Colloids Surf. 1992, 64, 299-310;

Diftis D.; Kiosseoglou V. Improvement of emulsifying properties of soybean proteinisolate by conjugation with carboxymethyl cellulose. Food Chemistry 2003, 81, 1-6;

Dunlap, C. A.; Côté, G. L. β-lactoglobulin-dextran conjugates: effect of polysaccharide size on emulsion stability. J. Agric. Food Chem. 2005, 53, 419-423;

Kato A.; Murata K.; Kobayashi K. Preparation and characterization of ovalbumindextranconjugate having excellent emulsifying properties. J. Agric.Food Chem. 1988, 36, 421-425;

Kato A.; Sato T.; Kobayashi K. Emulsifying properties of protein-polysaccharide complexes and hybrids. Agric. Biol. Chem. 1989, 53, 2147-2152;

Kato, A. Industrial applications of Maillard-type protein-polysaccharide conjugates. Food Sci. Technol. Res. 2002, 8, 193-199; and

Neirynck N.; Van der Meeren P.; Gorbe S. B.; Dierckx S.; Dewettinck K. Improved emulsion stabilizing properties of whey protein isolate by conjugation with pectins. Food Hydrocolloids 2004, 18, 949-957.

It has been reported recently that whey protein (WPI)-polysaccharide conjugates could be produced in aqueous solutions under “molecular crowding” conditions by incubating a mixture of concentrated WPI and polysaccharide solution at 60° C., at pH 6.5, for 48 h. Zhu, D.; Damodaran, S.; Lucey, J. A. Formation of whey protein isolate (WPI)-dextran conjugate in aqueous solutions. J. Agric.Food Chem. 2008, 56, 7113-7118. The purified WPI-dextran conjugate so produced was white in color and exhibited better heat stability in the pH range 3 to 7.5, and better emulsifying properties than unmodified WPI. (Zhu, D.; Damodaran, S.; Lucey, J. A. Physicochemical and emulsifying properties of whey protein isolate (WPI)-dextran conjugates produced in aqueous solution. J. Agric.Food Chem. 2010, 58, 2988-2994.) However, a major shortcoming of the above method is that the yield of the protein-polysaccharide conjugate was less than 10% even under molecular crowing conditions. The low yield might be due to the reversible nature of the Schiff base formation in aqueous solutions. Thus, an alternate strategy is needed to produce protein-polysaccharide conjugate to improve the functional properties of underutilized food proteins.

### SUMMARY

Disclosed herein is a compound (a conjugate) comprising a protein moiety covalently bonded to a polysaccharide moiety via a phosphate bridge. The preferred manner of making the conjugate is by reacting a mixture of a protein with a polysaccharide in the presence of POCl₃, for a time, a temperature, and a pH wherein the conjugate is formed. By way of example and not limitation, a conjugate as disclosed herein can be made by reaction whey protein isolate and dextran with POCl₃ at pH 10-10.5. This results in the formation of protein-phosphate-polysaccharide (PPP) conjugates cross-linked via a phosphate bridge. Molecules of this genus are referred to herein as “PPP conjugates.” The extent of conjugation increased with the weight ratio of protein to POCl₃ used in the reaction. This was confirmed by staining SDS-PAGE gels with Coomassie Blue G-250 for proteins and PAS reagent for carbohydrates. ³¹P NMR of the PPP conjugates also confirmed the presence of —N^{pro}—PO₂⁻—O^{Dex} cross-links in the PPP conjugate. Quantitative analysis of the ³¹P NMR revealed that about 32% of the phosphorylated lysine residues in the PPP conjugate were involved in the —N^{pro}—PO₂⁻—O^{Dex} bond. Fluorescence and CD spectroscopic analysis showed that while the conjugation caused changes in the tertiary structure, it did not significantly affect the secondary structure contents of the protein portion of the conjugate. Incubation of the PPP conjugate at pH 1.5 at 40° C. caused time-dependent hydrolysis of all N-phosphate bonds, including the —N^{pro}—PO₂⁻—O^{Dex} cross-links, and release of free amine groups. This indicated that lysine residues cross-linked to the polysaccharide in the PPP conjugate will be biologically available during transit through the gastro-intestinal tract.

It is shown herein that proteins contain hydroxyl groups (serine and threonine residues), ε-amino groups (in lysine residues), and himidazole group (on histidine residues) that can react with POCl₃. Polysaccharides contain several hydroxyl groups that can also react with POCl₃. In dilute solutions, reaction of POCl₃ with polysaccharides and proteins generally produces N- and O-phosphorylated proteins, and O-phosphorylated polysaccharides. (Zhu, D.; Damodaran, S. Chemical Phosphorylation Improves the Moisture Resistance of Soy Flour-Based Wood Adhesive. J. Appl. Polym. Sci. 2014, 131, 40451.) However, in a reaction mixture of protein and polysaccharide, these reactions can be shifted to produce protein-phosphate-polysaccharide (PPP) conjugate, in which POCl₃ acts as a bi-valent cross-linking reagent. This is shown schematically in FIG. 1. Unlike in conjugates formed via the Maillard reaction, the PPP conjugates are more anionic than the native protein due to additional negative charges on the phosphate bridge as well as other potential N-phosphate and O-phosphate derivatives on the protein and polysaccharide components of the conjugate.

Thus, disclosed herein is a compound comprising a protein moiety covalently linked to a polysaccharide moiety via a phosphate bridge. The protein moiety may comprise a whey protein. The polysaccharide moiety may comprise glucose residues. The phosphate bridge may be bound to an oxygen atom on the protein moiety, a nitrogen atom on the protein moiety, or both. The polysaccharide moiety may comprise dextran. In all versions of the compound, the protein moiety, the polysaccharide moiety, or both, may be pharmacologically active or nutritionally significant.

Also disclosed herein is a method of making a conjugate having a protein moiety and a saccharide moiety linked by a phosphate bridge, the method comprising contacting, in a solvent, a protein, a polysaccharide, and POCl₃ for a time, at a temperature, and at a pH wherein a reaction occurs that yields a protein-phosphate bridge-polysaccharide (“PPP”) conjugate. The solvent may be water. The reaction generally takes place at neutral to alkaline pH, from about pH 8 to about pH 14, or from about pH 8 to about pH 12, or from about pH 9 to about pH 11, or from about pH 10 to about pH 11.

It is generally preferred that in all versions of the method, the protein is present in a concentration of from about 5% (w/v) to about 50% (w/v) with respect to the solvent, and the polysaccharide is present in a concentration of from about 5% (w/v) to about 50% (w/v) with respect to the solvent. However, concentrations above and below these stated ranges are explicitly within the scope of the disclosed method. It is also generally preferred that the POCl₃ is present at a protein-to-POCl₃ ratio (w/w) of from about 0.5 to about 5. Again, this is a preferred range; concentrations above or below this ratio are explicitly within the scope of this disclosure.

The reaction is preferably conducted at a temperature of from about 10° C. to about 30° C. Temperatures above or below this range is also within the scope of the method.

The conjugates disclosed herein are useful in a number of applications. For example, they can be used as food-grade emulsifying agents. Emulsifying agents are extensively used in commercial food products as foaming agents, texture modifiers, etc. They are also useful to improve the solubility and stability of proteins in aqueous solutions—e.g., in protein drinks.

Specifically disclosed and claimed herein are:

1. A compound comprising a protein moiety covalently linked to a polysaccharide moiety via a phosphate bridge.

2. The compound of claim 1, wherein the polysaccharide moiety comprises a saccharide residue selected from the group consisting of ribose, arabinose, xylose, lyxose, allose, altrose, glucose, mannose, gulose, idose, galactose, talose, ribulose, xylulose, psicose, fructose, sorbose and tagatose.

3. The compound of claim 1, wherein the phosphate bridge is bound to an oxygen atom on the protein moiety.

4. The compound of claim 1, wherein the phosphate bridge is bound to a nitrogen atom on the protein moiety.

5. The compound of claim 1, wherein the polysaccharide moiety is dextran.

6. The compound of claim 1, wherein the protein moiety comprises a whey protein.

7. The compound of claim 6, wherein the polysaccharide moiety comprises a saccharide residue selected from the group consisting of ribose, arabinose, xylose, lyxose, allose, altrose, glucose, mannose, gulose, idose, galactose, talose, ribulose, xylulose, psicose, fructose, sorbose and tagatose.

8. The compound of claim 6, wherein the phosphate bridge is bound to an oxygen atom on the protein moiety.

9. The compound of claim 6, wherein the phosphate bridge is bound to a nitrogen atom on the protein moiety.

10. The compound of claim 6, wherein the polysaccharide moiety is dextran.

11. A method of making a conjugate having a protein moiety and a saccharide moiety linked by a phosphate bridge, the method comprising contacting, in a solvent, a protein, a polysaccharide, and POCl₃ for a time, at a temperature, and at a pH wherein a reaction occurs that yields a protein-phosphate bridge-polysaccharide (“PPP”) conjugate.

12. The method of claim 11, wherein the solvent is water.

13. The method of claim 11, wherein the pH is selected from a range of from about pH 8 to about pH 14, or from about pH 8 to about pH 12, or from about pH 9 to about pH 11, or from about pH 10 to about pH 11.

14. The method of claim 11, wherein the protein is present in a concentration of from about 5% (w/v) to about 50% (w/v) with respect to the solvent, and the polysaccharide is present in a concentration of from about 5% (w/v) to about 50% (w/v) with respect to the solvent.

15. The method of claim 11, wherein POCl₃ is present at a protein-to-POCl₃ ratio (w/w) of from about 0.5 to about 5.

16. The method of claim 11, wherein the temperature is from about 10° C. to about 30° C.

### BRIEF DESCRIPTION OF THE DRAWINGS

FIG. 1 is a schematic representation of protein-phosphate-polysaccharide conjugate formation under “molecular crowding” conditions according to the present disclosure.

FIG. 2 is a graph depicting the effect of POCl₃:WPI (w/w) ratio on the extent of phosphorylation/conjugation of lysine residues in a reaction mixture containing 10% WPI+20% dextran (♦), WPI alone (▪), and WPI-dextran 6 kDa mixture (▴).

FIGS. 3A and 3B are SDS-PAGE profile of WPI-dextran 6 kDa conjugates under reducing conditions. FIG. 3A is stained with Coomassie Blue G-250 for protein. FIG. 3B is stained with periodic acid-Schiff (PAS) reagent for carbohydrates. The conjugates were prepared using various WPI : POCl₃ ratios: Lanes al and b1, molecular weight markers; lanes a2 and b2, WPI; lanes a3 and b3, phosphorylated dextran 6 kDa control at 1:0.5 ratio; lanes a4 and b4, WPI-dextran conjugate at 1:0.5 ratio; lanes a5 and b5, phosphorylated dextran 6 kDa control at 1:1 ratio; lanes a6 and b6, WPI-dextran conjugate at 1:1 ratio; lanes a7 and b7, phosphorylated dextran 6 kDa control at 1:2 ratio; lanes a8 and b8, WPI-dextran conjugate at 1:2 ratio; lanes a9 and b9, phosphorylated dextran 6 kDa control at 1:3 ratio; lanes a10 and b10, WPI-dextran conjugate at 1:3 ratio.

FIGS. 3C and 3D are SDS-PAGE profiles of WPI-dextran 10 kDa conjugates under reducing condition. FIG. 3C was stained with Coomassie Blue G-250 for protein. FIG. 3D was stained with periodic acid-Schiff (PAS) reagent for carbohydrates. The conjugates were prepared using various WPI : POCl₃ ratios: Lanes c1 and d2, WPI; lanes c2 and d1, molecular weight markers; lanes c3 and d3, phosphorylated dextran 10 kDa control at 1:0.5 ratio; lanes c4 and d4, WPI-dextran 10 kDa conjugate at 1:0.5 ratio; lanes c5 and d5, phosphorylated dextran 10 kDa control at 1:1 ratio; lanes c6 and d6, WPI-dextran 10 kDa conjugate at 1:1 ratio; lanes c7 and d7, phosphorylated dextran 10 kDa control at 1:2 ratio; lanes c8 and d8, WPI-dextran 10 kDa conjugate at 1:2 ratio; lanes c9 and d9, phosphorylated WPI-dextran 10 kDa control at 1:3 ratio; lanes c10 and d10, WPI-dextran 10 kDa conjugate at 1:3 ratio.

FIG. 4A is a ³¹P NMR spectrum of phosphorylated dextran 6 kDa. FIG. 4B is a ³¹P NMR spectrum of phosphorylated WPI. FIG. 4C is a ³¹P NMR spectrum of WPI-dextran 6 kDa conjugate.

FIG. 5 is a graph showing the kinetics of regeneration of reactive lysine residues in WPI-dextran 6 kDa conjugate sample (●) and native WPI (▪) during incubation at pH 1.5 and 40° C.

FIG. 6 is a ³¹P NMR of dephosphorylated WPI-dextran 6 kDa conjugate.

FIG. 7 shows superimposed fluorescence spectra of native WPI (♦), WPI in 6M urea (▪), WPI-dextran conjugate produced using WPI:POCl₃ ratio (w/w) of 1:0.1 (▴), WPI-dextran conjugate produced using WPI:POCl₃ ratio (w/w) of 1:0.5 (◯), and WPI-dextran conjugate produced using WPI:POCl₃ ratio (w/w) of 1:2 (●). The excitation wavelength was 280 nm.

FIG. 8 shows superimposed CD spectra of native WPI (●), WPI phosphorylated at WPI:POCl₃ ratio of 1:1 (▴), and PPP conjugate (▪).

FIG. 9 shows superimposed pH-turbidity profiles for various WPI-dextran conjugates. WPI (♦); WPI:PoCl₃ (▪); WPI:Dex 6 kDa (▴); WPI:Dex 10 kDa (X); WPI:Dex 20 kDa (*).

FIGS. 10A, 10B, 10C, 10D, 10E, 10F, 10G, and 10H are a series of photographs showing the visual appearances of the turbidity of WPI and WPI-dextran conjugates at pH 4.56 before and after centrifugation at 11000 g for 2 min. FIGS. 10A and 10B: native WPI before and after centrifugation, respectively. FIGS. 10C and 10D: WPI:Dextran (6 kDa) conjugate before and after centrifugation, respectively. FIGS. 10E and 10F: WPI:Dextran (10 kDa) conjugate before and after centrifugation, respectively. FIGS. 10G and 10H: WPI:Dextran (20 kDa) conjugate before and after centrifugation, respectively.

FIG. 11 is a graph showing pH versus Zeta potential profile of WPI-dextran (6 kDa) conjugate at various extents of phosphorylation. The WPI to POCl₃ ratios were: 1:0.0 (control, ●), 1:0.5 (▴), 1:1 (▪), 1:2 (♦), Dextran (6 kDa) control phosphorylated at dextran to POCl₃ ratio of 1:2 (◯).

### DETAILED DESCRIPTION

### Abbreviations and Definitions:

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations as used herein shall include the corresponding plural characteristic or limitation, and vice-versa, unless otherwise specified or clearly implied to the contrary by the context in which the reference is made. That is, unless specifically stated to the contrary, “a” and “an” mean “one or more.” The phrase “one or more” is readily understood by one of skill in the art, particularly when read in context of its usage. For example, “one or more” substituents on a phenyl ring designates one to five substituents.

All combinations of method or process steps as used herein can be performed in any order, unless otherwise specified or clearly implied to the contrary by the context in which the referenced combination is made.

The methods of this disclosure can comprise, consist of, or consist essentially of the essential elements and limitations of the method described herein, as well as any additional or optional ingredients, components, or limitations described herein or otherwise useful in synthetic organic chemistry.

The term “about” is used herein generally to mean a value±5% of a given numerical value. Thus, “about 60%” refers to a value of 60±5% of 60 (i.e., between 57 and 63).

The term “contacting” refers to the act of touching, making contact, or of bringing to immediate or close proximity, including at the molecular level, for example, to bring about a chemical reaction, or a physical change, e.g., in a solution or in a reaction mixture.

An “effective amount” refers to an amount of a chemical or reagent effective to facilitate a chemical reaction between two or more reaction components, and/or to bring about a recited effect. Thus, an “effective amount” generally means an amount that provides the desired effect.

As used herein, the terms “phosphodiester bond” and “phosphate bridge” are synonymous and refer to a divalent linkage having the structure R—P(O₂)⁻—R′, wherein R and R′ can be the same or different.

The terms “saccharide” and “monosaccharide” refer to compounds of the molecular formula Cₙ,H₂ₙOₙ, wherein “n” is an integer of from 3 to 7, and all isomers thereof, without limitation. Saccharides include aldoses and ketoses. A non-limiting list of examples includes ribose, arabinose, xylose, lyxose, allose, altrose, glucose, mannose, gulose, idose, galactose, talose, ribulose, xylulose, psicose, fructose, sorbose and tagatose. The term “polysaccharide refers to an oligomer or polymer of saccharide residues, and all isomers, anomers, and epimers thereof.

The term “solvent” refers to any liquid that can dissolve a compound to form a solution. Solvents include water and various organic solvents, such as hydrocarbon solvents, for example, alkanes and aryl solvents, as well as halo-alkane solvents. Examples include hexanes, benzene, toluene, xylenes, chloroform, methylene chloride, dichloroethane, and alcoholic solvents such as methanol, ethanol, propanol, isopropanol, and linear or branched (sec or tert) butanol, and the like. Aprotic solvents that can be used in the method include, but are not limited to perfluorohexane, α,α,α-trifluorotoluene, pentane, hexane, cyclohexane, methylcyclohexane, decalin, dioxane, carbon tetrachloride, freon-11, benzene, toluene, triethyl amine, carbon disulfide, diisopropyl ether, diethyl ether, t-butyl methyl ether (MTBE), chloroform, ethyl acetate, 1,2-dimethoxyethane (glyme), 2-methoxyethyl ether (diglyme), tetrahydrofuran (THF), methylene chloride, pyridine, 2-butanone (MEK), acetone, hexamethylphosphoramide, N-methylpyrrolidinone (NMP), nitromethane, dimethylformamide (DMF), acetonitrile, sulfolane, dimethyl sulfoxide (DMSO), propylene carbonate, and the like.

CD=circular dichroism. NMR=nuclear magnetic resonance. PAS=periodic acid (H₅IO₆/HIO₄)-Schiff reagent (or stain). PPP conjugates=a molecule comprising a protein moiety covalently linked to a polysaccharide moiety via a phosphate bridge. SDS PAGE=sodium dodecyl sulfate polyacrylamide gel electrophoresis. TNBS=trinitrobenzenesulfonic acid. WPI=whey protein isolate.

Materials: Whey protein isolate (WPI) samples were provided by Agropur Ingredients (Minneapolis, Minn., USA). The sample contained 97.9% protein and 4.7% moisture. The various molecular weight dextrans (6,000, and 10,000 Da) from Leuconostoc spp, were purchased from Alfa Aesar (Tewksbury, Mass., USA). 2,4,6-Trinitrobenzenesulfonic acid (TNBS), Pierce glycoprotein staining kit for carbohydrate staining, and pre-stained molecular weight markers (EZ-run™) were purchased from Thermo Fisher Scientific (Waltham, Mass., USA). POCl₃ (CAS No. 10025-87-3) was purchased from Millipore Sigma (Burlington, Mass. USA).

Synthesis of WPI-Dextran Conjugates: Molecular crowding conditions were created by dissolving 10% (w/v) WPI and 20% (w/v) dextran in water. The solution was stirred on a magnetic stirrer at room temperature until the solution was homogeneous. The pH of the solution was adjusted to 10.5. The protein and the polysaccharide were cross-linked via a phosphate bridge by reacting with POCl₃. A calculated amount of POCl₃ was added in small (μL) aliquots to the protein-polysaccharide mixture solution over a period of up to one hour with vigorous stirring. The pH of the solution was continuously maintained between about 10 and about 10.5 during the reaction by adding small amounts of concentrated base (10 M NaOH). The extent of conjugation was varied by varying the protein-to-POCl₃ ratio (w/w) from 0 to 3. Once the reaction was completed, the solution was stirred for an additional 15 min at about pH 10 to about pH 10.5 and then the pH was adjusted to pH 7. Because the final volumes of the reacted solutions were different at different protein:POCl₃ ratios (1:0.5, 1:1, 1:2, and 1:3), all the solutions were made up to a same final volume to avoid variations in protein and dextran concentrations. Small aliquots (2 mL) of these samples were withdrawn and stored frozen for subsequent chemical and electrophoretic analyses. The rest of the samples were dialyzed using either 6-8 kDa molecular weight cut-off membrane for 72 h at 4° C. to remove salts (NaCl and Na₃PO₄) formed during the reaction. The samples were then lyophilized and stored at 4° C. for future use.

Phosphorylated WPI and dextran controls also were prepared in a similar manner. Briefly, 10% (w/v) WPI and 20% (w/v) dextran solutions were phosphorylated separately using same amounts of POCl₃ as were used in the above WPI-polysaccharide mixture reactions. After the reaction, the volumes of these phosphorylated control solutions were also made up to a final volume as the above samples; small (2 mL) aliquots these samples were withdrawn and stored frozen for subsequent chemical and electrophoretic analyses and the rest of the solutions were dialyzed as above and freeze dried and stored at 4° C. for future use.

Determination of Protein and Dextran Contents: Protein estimation using the Biuret method was not possible with protein-dextran samples as the dextran precipitated upon the addition of the Biuret reagent. Therefore, the protein content of the samples was determined using the 205/280 nm absorbance method using the following equation:

$$Protein content ( mg / mL ) = \frac{{A}_{205}}{⌊ 27 + 120 ( \frac{{A}_{280}}{{A}_{205}} ) ⌋}$$

where A₂₀₅ is the absorbance at 205 nm and A₂₈₀ is the absorbance at 280 nm. See Scopes, R. K. Measurement of protein by spectrophotometry at 205 nm, Analytical Biochemistry 1974, 59, 277-252 and Whitaker, J. R. and Granum, P. E. An Absolute method for protein determination based on difference in absorbance at 235 and 280 nm. Analytical Biochemistry 1980, 109, 156-159. Because dextran absorbs at 205 nm (but not at 280 nm), the final protein content was determined by quantitatively subtracting the A₂₀₅ contribution from dextran. The phenol-sulfuric acid method (Dubois et al., 1956) was used to determine the concentration of dextran in samples. Dubois, M. et al. Colorimetric Method for determination of sugars and related substances. Analytical Chemistry 1956, 28, 350-356.

Sodium Dodecyl Sulfate-Polyacrylamide Gel Electrophoresis (SDS-PAGE): Gel electrophoresis was done on a “Mini-Protean”®-brand device (Biorad Laboratories, Mass., USA) using a 4% stacking and 12% resolving gel under reducing conditions according to the method of Laemmli (1970). Two gels were run simultaneously at 200 mV for 55 minutes. After electrophoresis, one of the gels was stained with Coomassie blue G-250 and the other was stained with PAS glycoprotein staining kit. See Laemmli, U. K. Cleavage of structural proteins during the assembly of the head of bacteriophage T4. Nature 1970, 227, 680-685.

Lysine Estimation: Initial experiments to determine the lysine content of protein-dextran conjugates using the method of Hall et al (1956) was unsuccessful due to caramelization of dextran during acid hydrolysis at 100° C. Therefore, the method of Habeeb (1966), with modifications, was used to determine the lysine content of protein-dextran conjugates. Briefly, to 1 mL of sample solution containing 200 μg protein was added 1 mL of 1M NaHCO₃ (pH 8.5) and 1 mL of 0.1% TNBS. The modification involved use of 1M NaHCO₃, as opposed to 4% NaHCO₃ originally proposed (Habeeb (1966)), to ensure that the pH was maintained at 8.5 throughout the reaction. The contents were incubated at 40° C. for 2 h. The reaction mixture was then mixed with 3.5 mL of concentrated HCl and the absorbance was measured at 415 nm. The lysine estimation was done in triplicates. See Hall, R. J. et al. Observations on the Use of 2,4,6-trinitrobenzenesulphonic acid for the determination of available lysine in animal protein concentrates. Analyst, 1973, 98, 673-686 and Habeeb, A. F. S. A. Determination of free amino groups in proteins by trinitrobenzenesulfonic acid. Analytical Biochemistry 1966, 14, 328-336.

Dephosphorylation: The time course of dephosphorylation of the protein-dextran conjugates under acidic condition was studied as follows: The pH of protein-dextran conjugate solution (20 mL) containing 0.2 mg/mL protein concentration was adjusted to 1.5 and incubated at 40° C. Aliquots (1 mL) were withdrawn at 15 min intervals for the first 2 h and at 30 min intervals for the next 2 h and were subjected to lysine determination, as described above. A control consisting of unmodified WPI at the same protein concentration as in the protein-dextran conjugate sample was treated in the same manner and its lysine content as a function of incubation time at pH 1.5 and 40° C. was determined. These measurements were done in duplicates.

Fluorescence Measurements: Fluorescence spectroscopic measurements were done using Perkin Elmer LS-5B luminescence spectrometer (Perkin Elmer, Billerica, Mass., USA) to study conformational changes in the protein upon conjugation with polysaccharide.

Zeta Potential Measurements: Zeta potential of protein-polysaccharide conjugate were determined at various pHs using a 90 Plus-brand particle analyzer (Brookhaven Instruments Corp., N.Y., USA).

pH-Turbidity Profile: Solutions containing 0.8 mg/mL protein-polysaccharide conjugate were heat denatured by incubating in a boiling water bath for 10 min and then cooled to room temperature by immersing in a cold-water bath. Aliquots (2 mL) of the solution were then adjusted to various pHs and were allowed to stand at room temperature for 15 minutes, after which the turbidity of the solution was measured at 600 nm in a UV-visible spectrophotometer (Shimadzu UV-P1601 PC, Shimadzu Corp., Kyoto, Japan). A control solution containing a physical mixture of WPI and polysaccharide at same concentrations as found in the protein-polysaccharide conjugates was subjected to same treatment as the samples and its pH-turbidity profile was determined at 600 nm. These measurements were made in triplicates.

³¹P NMR spectroscopy: Proton-decoupled ³¹P NMR was acquired on a Bruker Avance III 600 NMR spectrometer (240 MHz for ³¹P) equipped with a 5 mm cryogenic probe at the National NMR Facility at the University of Wisconsin-Madison, Madison, Wis., USA. The spectra were acquired with 32,768 scans over a period of 24 h at 298 K. The Bruker pulse sequence of zgpg30 was used. Phosphoric acid was used as the internal standard.

Circular Dichroism (CD) Spectroscopy: The secondary structure of the protein was studied using a Biorad Proteon XPR36 model circular dichroism spectrometer (Biorad, Mass., USA). Measurements were done at 1 mm path length with a 0.2% protein concentration. The raw data (in mdeg) were converted to the mean residual ellipticity (deg.cm²,dmol⁻¹) using the following equation (Damodaran, 1989):

[θ]=100dM/C

where d is rotation in degrees/cm path length, M is the average molecular weight of amino acid residues in the protein (=115 Da), and C is protein concentration in mg/mL. (Damodaran, S. Influence of protein conformation on its adaptability under chaotropic conditions. Int. J. Biol. Macromol, 1989, 11, 2-8.) Secondary structure estimates from CD spectra were made using the CDESTIMA program. (Chang, C. T.; Wu, C. S. C.; Yang, J. T. Circular dichroic analysis of protein conformation: inclusion of the beta-turns. Anal. Biochem. 1978, 91, 13-31.)

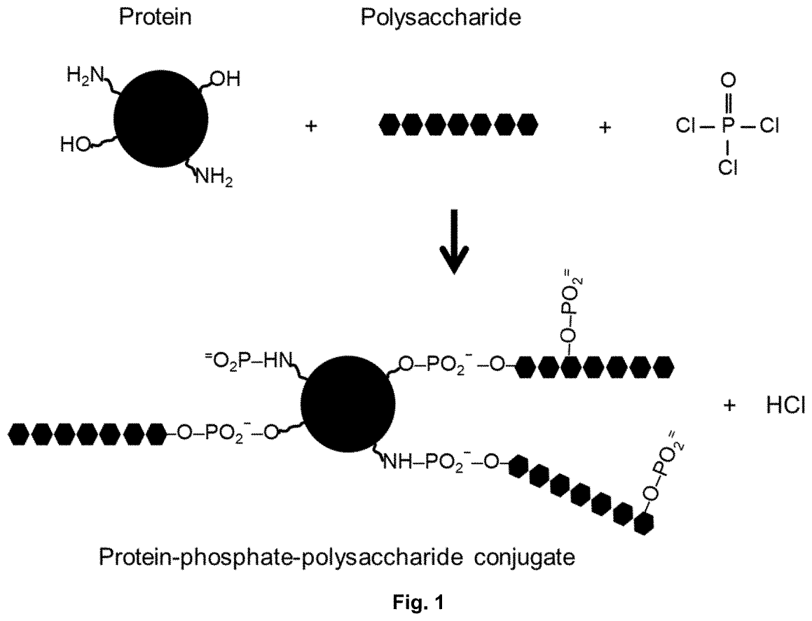

Extent of Conjugation/Phosphorylation: WPI and dextran contain several reactive groups that can react with POCl₃ at pH 10.0 to 10.5. These include the amine groups of lysine and histidine, the hydroxyl groups of serine and threonine residues in WPI, and the hydroxyl groups in dextran (located at positions 2, 3, and 4 of the glucose monomers). Thus, the phosphorylation/cross-linking reaction between protein and polysaccharide can occur between any set of these reactive groups. FIG. 1 shows this schematically, as the phosphorous atom can both N-link and O-link to the protein. This makes it difficult experimentally to follow all reaction paths. In this investigation, the extent of phosphorylation/conjugation was determined in terms of percent decrease in reactive lysine residues in the protein after the phosphorylation reaction.

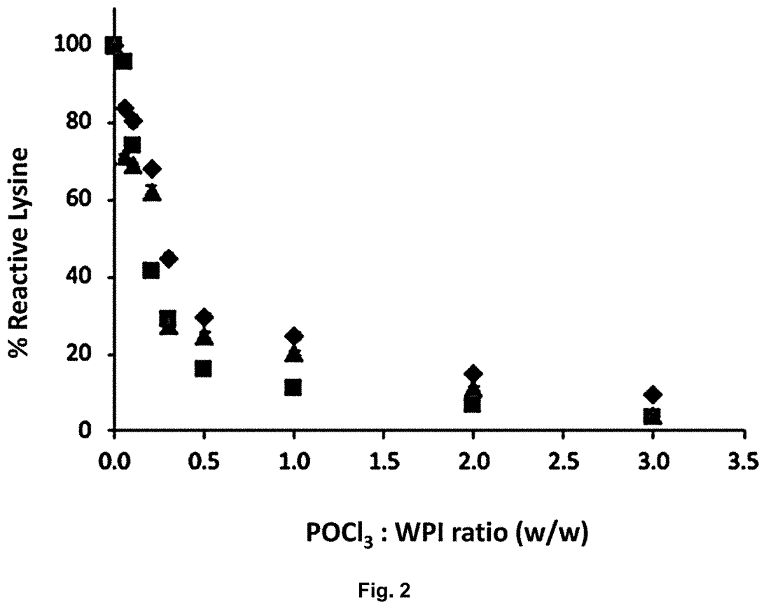

The extent of phosphorylation/conjugation in WPI (10%)+dextran (20%) mixtures as a function of the weight ratio of POCl₃ to WPI used in the reaction is shown in FIG. 2 for 6 kDa and 10 kDa dextrans. The phosphorylation reaction with 10% WPI also is shown in FIG. 2 for comparison. The available lysine content of the protein decreased sharply to about 20% as the POCl₃ to WPI ratio (w/w) was increased from zero to 1 and thereafter it decreased asymptotically toward zero as the ratio was increased to 3. No significant difference in the reaction profile between WPI and WPI+dextran mixtures was evident. However, because molecular crowding in 10% WPI+20% dextran mixtures would be much higher than in 10% WPI alone, it is likely that the nature of the products formed in these two cases might be different: That is, the probability of heterologous (as well as homologous) cross-linking reactions would be more in the 10% WPI+20% dextran mixtures than in 10% WPI alone.

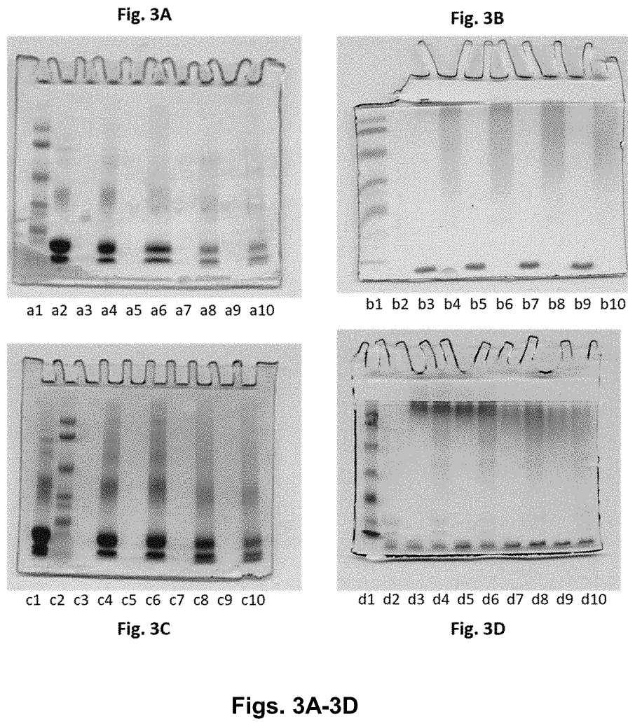

SDS-PAGE of WPI-Dextran Conjugates: The formation of WPI-dextran conjugates through a phosphate bridge (a type of PPP conjugate) in the above samples was analyzed by SDS-PAGE under reducing conditions. See FIGS. 3A, 3B, 3C, and 3D. The gels were individually stained with Coomassie Blue G-250 to detect protein bands (FIGS. 3A and 3C) and with periodic-acid Schiff reagent (PAS) to detect carbohydrates and PPP conjugates (FIGS. 3B and 3D). In the Coomassie blue-stained gels, the unmodified WPI profile showed bands for α-lactalbumin at 14 kDa, β-lactoglobulin at 18 kDa, and bands at 38.7 kDa and 66 kDa (FIGS. 3A and 3C). The intensities of these protein bands progressively decreased as the protein-to-POCl₃ ratio used in the reaction was increased from 1:0 to 1:3, with concomitant appearance of a high molecular mass diffused band with molecular weight in the range of 120-18 kDa stretching down from top of the separating gel. The phosphorylated, dextran-only controls were not stained by Coomassie Blue G-250 and those lanes appeared blank in these gels. Because these gels were run under reducing conditions, the high molecular mass diffused band ought to be protein polymers formed by cross-links other than disulfide cross-links. It has been observed that phosphorylation decreased the binding of Coomassie Blue G-250 to protein, presumably because of charge-charge repulsion between the negatively charged dye and the negatively charged phosphorylated protein (and conjugate). As a result, the diffused bands in FIGS. 3A and 3C appear faint.

FIGS. 3B and 3D show the SDS-PAGE profiles stained with PAS reagent for detecting carbohydrate-containing proteins. The lanes corresponding to control WPI were not stained by PAS in these gels, whereas the lanes loaded with PPP conjugates were stained by PAS. The pink-colored bands were diffused with compounds having molecular masses ranging from 120 kDa to 18 kDa, very similar to the diffused protein bands in FIGS. 3A and 3C. This strongly indicates that the diffused high molecular mass bands in the Coomassie Blue-stained gels (FIGS. 3A and 3C) were PPP conjugates. Dextran, which is an α-1,6-D-glucose polymer, is neutral, hydrophilic, and does not bind SDS. Therefore, dextran itself does not migrate in SDS-PAGE. When dextran is phosphorylated it can migrate in SDS-PAGE. But, because of its inability to bind SDS, the charge per unit mass of phosphorylated dextran will be less than that of proteins in SDS-PAGE. As a result, phosphorylated dextran does not move based on its molecular mass in SDS-PAGE as proteins do. When dextran (and phosphorylated dextran) is covalently cross-linked to protein, the conjugate will have a greater electrophoretic mobility than dextran itself (which has none) or phosphorylated dextran (which has some). This is evident from the electrophoresis profiles or PPP conjugates in FIGS. 3B and 3D. For instance, in lanes corresponding to phosphorylated dextran-10 kDa in FIG. 3D, the phosphorylated dextran migrates into the stacking and separating gels; the migration is greater with the degree of phosphorylation. However, in lanes corresponding to PPP conjugates, the staining is more intense and the migration is much farther than in corresponding phosphorylated dextran-alone lanes. This difference in intensity and broadening of the diffused band arises because of PPP conjugation. It should be noted that, unlike in FIG. 3D, the lanes corresponding to phosphorylated dextran-6 kDa in FIG. 3B are blank. This suggests that because of its small size, the phosphorylated dextran-6 kDa molecules had leached out of the gel during destaining; whereas in the case of dextran-10 kDa (FIG. 3D), because of its larger size, the phosphorylated dextran 10 kDa was partially trapped in the SDS-PAGE gel.

³¹P NMR: To obtain further corroborative evidence that protein—PO₄⁻—polysaccharide cross-links are formed during the reaction with POCl₃, the proton-decoupled ³¹P NMR spectra of the PPP conjugate were studied. Phosphorylated WPI and phosphorylated dextran were used as controls to identify the chemical shift attributable to protein—PO₄⁻—polysaccharide cross-links.

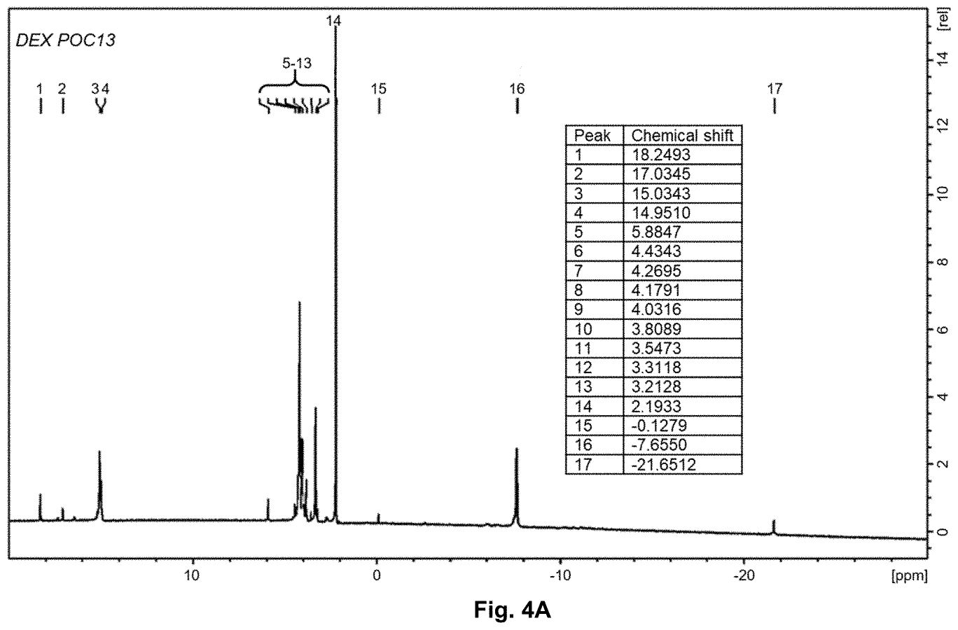

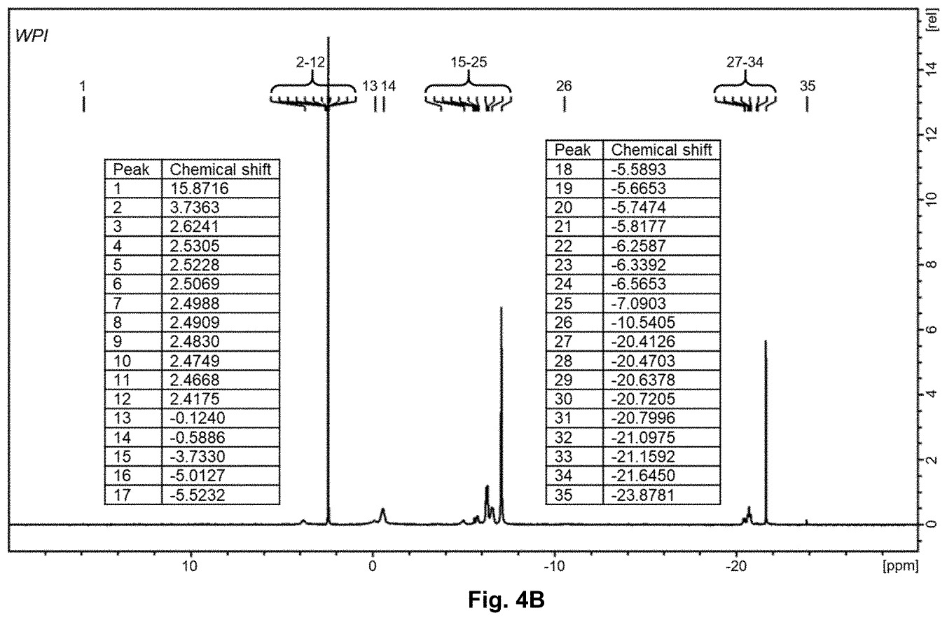

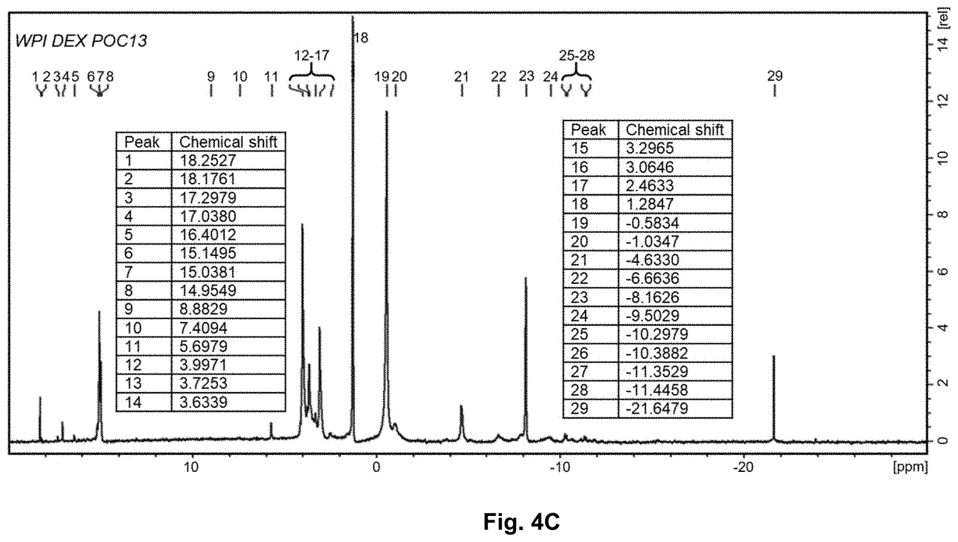

The ³¹P NMR of phosphorylated dextran (6 kDa) showed chemical shifts at four different regions, namely, at 18.2493 to 14.9510 ppm, 5.8847 to 2.1904 ppm, -7.6650 ppm, and −21.6512 (FIG. 4A). Because dextran is an a-(1→6)-D-glucose polymer, there is only one C-6 hydroxyl group at the non-reducing end of dextran molecule. Thus, the hydroxyl groups at positions 2, 3, and 4 of the glucose units are the primary sites of phosphorylation/conjugation. Among these, the C-2 hydroxyl group is considered the first to react. (Allene, J. Dextran. “In Encyclopedia in polymer science and engineering technology,” Mark, H. F.; Gaylord, N. G.; Bikales, N. M., (Eds.); pp 805-824, John Wiley & Sons, New York, 1966.) The region at 5.8847 to 2.1904 ppm could be assigned to the dextran phosphate (—O—PO₃²⁻) of hydroxyl groups at positions 2, 3, and 4. See Suflet et al., Phosphorylated polysaccharides. 2. Synthesis and properties of phosphorylated dextren. Carbohydrate Polym. 2010, 82, 1217-1277 and Sacco et al., Re-investigation of the phosphorylation of dextran with polyphosphoric acid: Evidence for the formation of different types of phosphate moieties. Carbohydrate Res. 1988, 184, 193-202. The minor peaks at 18.2493 to 14.9510 ppm could be attributed to —O—PO₃²⁻ at the C-6 hydroxyl group at the non-reducing end of dextran, where de-shielding would be much more than at the 2, 3, and 4 positions. The peaks at −7.6650 and −21.6512 ppm, which are highly shielded, could be attributed to diphosphates of the kinds —O—PO₂^{-—PO}₃H and triphosphate, i.e., —O—PO₂⁻—PO₂⁻—PO₃H, respectively attached to dextran hydroxyl groups. (Matheis, G.; Whitaker, J. R. ³¹P NMR chemical shifts of phosphate covalently bound to proteins. Int. J. Biochem. 1984, 16, 867-873.)

The ³¹P NMR of phosphorylated WPI showed four distinct chemical shift regions, at 2.48, −0.5886, −6.2587 to −7.0903, and −20.6378 to −21.645 ppm (FIG. 4B). The 2.48 ppm could be assigned to the N-phosphate (—NH—PO₃²⁻) at the lysine residue. This is similar to the —O—PO₃²⁻ peak at 2.19 ppm in phosphorylated dextran (FIG. 4A). It has been reported that the ³¹P NMR of phospho-polylysine contained two peaks, one at −1.28 ppm and the other at 4.51 ppm relative to orthophosphoric acid reference (Mathies and Whitaker, 1984, supra). Because shielded phosphate usually has a negative chemical shift, the peak at −1.28 ppm in phospho-polylysine likely belongs to the —NH—PO₂⁻—NH— cross-link between polylysine molecules. Likewise, the minor peak at −0.5886 ppm in phosphorylated WPI likely belongs to —NH—PO₂⁻—NH— and —NH—PO₂⁻—O— cross-links between protein molecules. The slight upper/lower shifts of these peaks in phosphorylated WPI compared to those in phospho-polylysine might be due to variations in local microenvironments in these two systems. The peaks at −6.2587 to −7.0903 in phosphorylated WPI belong to diphosphates of the kind —O—PO₂⁻—PO₃²⁻ and —NH—PO₂⁻—PO₃²⁻, and the peaks at −20.6378 to −21.645 ppm belong to triphosphate derivatives (Sacco et al., 1988; Mathies and Whitaker, 1984, supra), indicating that significant amount of protein-linked diphosphates and triphosphates were produced during the reaction of POCl₃ with WPI at 10% (w/v) concentration.

The ³¹P NMR of the PPP conjugate showed peaks corresponding to those present in phosphorylated dextran and phosphorylated WPI, and three additional peaks arising from PPP conjugation (FIG. 4C). As noted earlier, the peaks in the region 6 to 2.2 ppm belong to —O^{Dex}—PO₃²⁻. The relative intensity of these peaks has reduced from 56% in the dextran phosphate to about 37% in the PPP conjugate sample. This reduction can be attributed to competition between WPI and dextran for reaction with POCl₃. Three new peaks at 1.2647 ppm, −0.5834 ppm, and −4.633 ppm are seen in the PPP conjugate. The major new peak at 1.2647 ppm in the PPP conjugate, which is down-shifted compared to the 2.48 ppm peak in phosphorylated WPI (FIG. 4B), belongs to monophosphates —N^{pro}—PO₃²⁻ and —O^{pro}—PO₃²⁻. The down-shifting is likely due to relatively more restricted local environment in the PPP conjugate than in phosphorylated WPI. The major new peak at −0.5834 ppm and its doublet at −1.0347 ppm, which was present as a very minor peak in phosphorylated WPI at −0.5886 ppm (FIG. 4B) is relatively more shielded than the monophosphates. This peak can be attributed to —N^{pro}—PO₂⁻—O^{Dex}— and —O^{pro}—PO₂⁻—O^{Dex}— arising from cross-linking of dextran with lysine residues and/or cross-linking of dextran with serine hydroxyl groups, respectively. Alkyl diphosphates and triphosphates usually exhibit chemical shifts in the region −5 to −9 and at >−20 ppm, respectively (Sacco, et al., 1988; Matheis and Whitaker, 1984, supra). Thus, the peaks at −4.7191 ppm can be attributed to diphosphates of the kind —O^{pro}—PO₂⁻—PO₃²⁻ and —N^{pro}—PO₂⁻—PO₃²⁻, and the peak at −21.6479 ppm could be triphosphates —N^{pro}—PO₂⁻—PO₂⁻—PO₃²⁻ and —O^{pro}—PO₂⁻—PO₂⁻—PO₃²⁻. The peak assignments for various phosphate groups found in phosphorylated dextran, WPI, and PPP conjugate is summarized in Table 1. In summary, the new major peak at −0.5834 ppm and its doublet at −1.0347 ppm in the WPI-dextran conjugate sample are indicative of formation of protein-phosphate-dextran cross-links. This corroborates well with the SDS-PAGE results.

TABLE 1
³¹P NMR peak assignments for phosphorylated WPI, phosphorylated
dextran, and WPI-dextran conjugates

| | Chemical Shift | Peak |
|---|---|---|
| Sample | (ppm) | Assignment |
| Phosphorylated | 2.48 | —N^{pro}—PO₃²⁻ |
| WPI | −3.7 to −7.09 | —O^{pro}—PO₂⁻—PO₃²⁻ and |
| | | —N^{pro}—PO₂⁻—PO₃²⁻ |
| | −20.6378 to −21.6479 | —N^{pro}—PO₂⁻—PO₂⁻—PO₃²⁻ and |
| | | —O^{pro}—PO₂⁻—PO₂⁻—PO₃²⁻ |
| Phosphorylated | 18.2493 to 14.951 | —O⁶—PO₄²⁻ |
| dextran | 5.8847 to 2.1904 | —O—PO₃²⁻ |
| | | (at 2, 3, and 4^{th} positions) |
| PPP conjugate | 1.2647 | —N^{pro}—PO₃²⁻ and |
| | | —O^{pro}—PO₃²⁻ |
| | −0.5834 and −1.0347 | —N^{pro}—PO₂⁻—O^{Dex} |
| | −4.7191 and 8.16 | —N^{pro}—PO₂⁻—PO₃²⁻ and |
| | | —O^{pro}—PO₂⁻—PO₃²⁻ |
| | −21.6459 | —N^{pro}—PO₂⁻—PO₂⁻ PO₃²⁻ |

N^{pro} = amine group of protein; O^{per} = hydroxyl group of protein; and O^{Dex} = hydroxyl of dextran; WPI = Whey protein isolate.

De-Phosphorylation: Lysine is an essential amino acid. Both phosphorylation and conjugation of lysine residues in a protein to dextran might affect its biological availability unless it is dephosphorylated and the PPP conjugate is cleaved during transit through the gastro-intestinal tract. N-phosphate linkages are known to be prone to acid hydrolysis. Because the acidity in the human stomach ranges from roughly pH 1.5 to 4.0, it is possible that the —N^{pro}—PO₃²⁻ and —N^{pro}—PO₂⁻—O^{Dex}— linkages in the PPP conjugate might be cleaved during transit through the stomach.

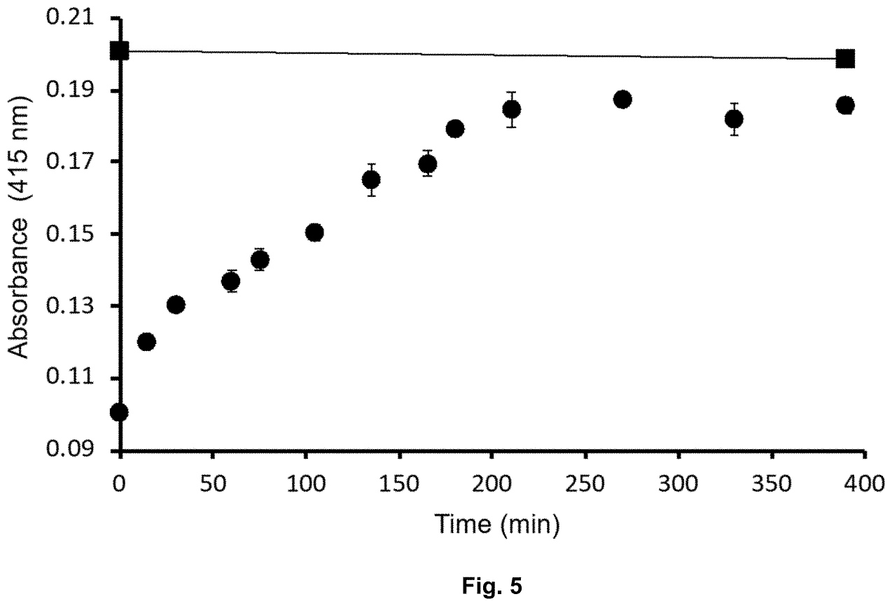

The time course of release of free amine groups in the PPP (10 kDa) conjugate during incubation at pH 1.5 and 40° C. is shown in FIG. 5. Data for unmodified WPI at the same protein concentration as in the PPP conjugate sample also is shown in FIG. 5. There was no change in the TNBS-reactive amine content of the WPI control over the entire incubation period of 400 min, indicating that there was no peptide hydrolysis and release of α-amino groups under these incubation conditions. Thus, the A₄₁₅ of the WPI control, which remained constant over a period of 400 min incubation time, represented the total TNBS-reactive lysine α-amine content of the control. In contrast, the A₄₁₅ value of the PPP conjugate sample increased with incubation time at pH 1.5, indicating a time-dependent release of TNBS-reactive α-amino groups. The A₄₁₅ reached a final value slightly lower than the upper limit for total TNBS-reactive lysine present in the control WPI, suggesting that not all N-phosphate species in the PPP conjugate were dephosphorylated at pH 1.5. Nevertheless, the data clearly indicated that a majority (˜90%) of phosphorylated/conjugated lysine residues in the PPP conjugate were released at the pH conditions of the acidic stomach. However, because the transit time of food through the acidic stomach is typically about 60 min (Dupont, et al., Comparative resistance of food proteins to adult and infant in vitro digestion models. Mol. Nutr. Food Res. 2010, 54, 767-780), this release would be only partial according to the data in FIG. 5.

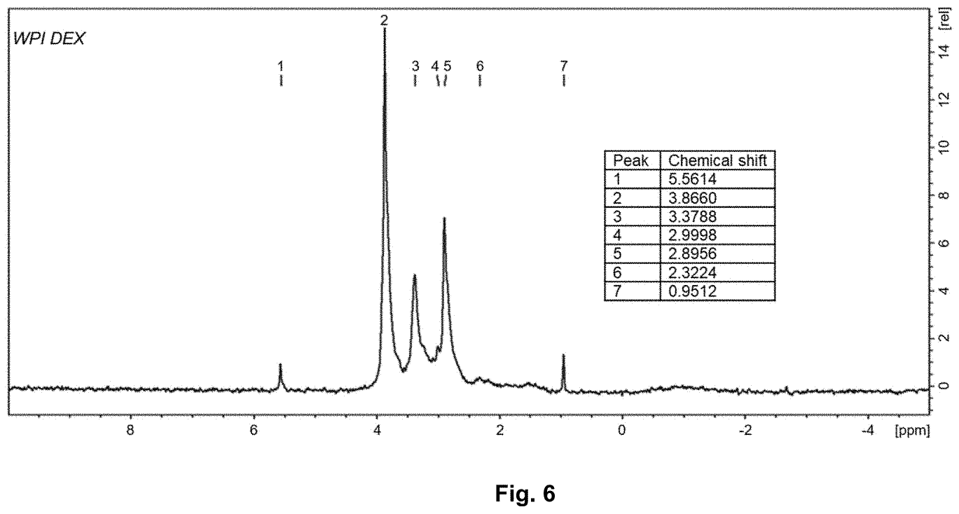

To identify which phosphate bonds in the PPP conjugate were broken during acid hydrolysis at pH 1.5, the ³¹P NMR of dephosphorylated PPP conjugate (400 min sample) was investigated. See FIG. 6. When compared to the ³¹P NMR of the intact PPP conjugate (FIG. 4C), the ³¹P NMR of dephosphorylated PPP conjugate showed peaks in the 3 to 6 ppm region, which belong to dextran phosphorylated at 2, 3, and 4 hydroxyl positions, and a minor peak at 0.95 ppm, which belongs to NH—PO₃²⁻ of protein. The major peaks at 1.2647, −0.5834, −4.7191, and −21.6459 ppm originally present in the PPP conjugate (FIG. 4C) were completely absent in the dephosphorylated PPP conjugate whose spectrums shown in FIG. 6. It has been reported that O-linked alkyl phosphates, such as methyl dihydrogen phosphate (—O—PO₃²⁻) and dimethyl phosphate (—O—PO₂⁻—O—) were stable to acid hydrolysis in the pH range 0.5-3.0. (Bunton, et al., The reactions of organic phosphates. Part III. The hydrolysis of dimethyl phosphate. J. Chem. Soc. 1960, pp. 3293-3301 and Bunton, et al., The reactions of organic phosphates. Part I. The hydrolysis of methyl dihydrogen phosphate. J. Chem. Soc. 1958, pp. 3574-3587.) This is evident also from the persistence of —O—PO₃²⁻ groups belonging to phosphorylated dextran (3 to 6 ppm) moieties in the dephosphorylated PPP conjugate (FIG. 6). This indicates that the ³¹P NMR peaks that disappeared upon dephosphorylation at pH 1.5 were all acid labile N-phosphate links of the kind —N^{pro}—PO₃²⁻, —N^{pro}—PO₂⁻—O^{Dex}, —N^{pro}—PO₂⁻—PO₃²⁻, and —N^{pro}—PO₂⁻—PO₂⁻—PO₃²⁻. Based on this analysis, it can be concluded that —N^{pro}—PO₂⁻—O^{Dex} (the peak at −0.5834 ppm) was the only type of cross-link present in the PPP conjugate. The fractional area of the peak at -0.5834 ppm out of all the peaks related to N-phosphorylated species in FIG. 4C was about 0.32, implying that about 32% of the phosphorylated NH₂ groups the PPP conjugate were in the form of —N^{pro}—PO₂⁻—O^{Dex} bond conjugated to dextran, and the remaining were in the form of amine mono, di, and tri-phosphates.

The minor peak at 0.95 ppm in the dephosphorylated PPP conjugate might belong to either a minor fraction of the —N^{pro}—PO₃²⁻ specie resistant to hydrolysis, presumably because of restricted accessibility, or it could be —O—PO₃²⁻ of serine residues.

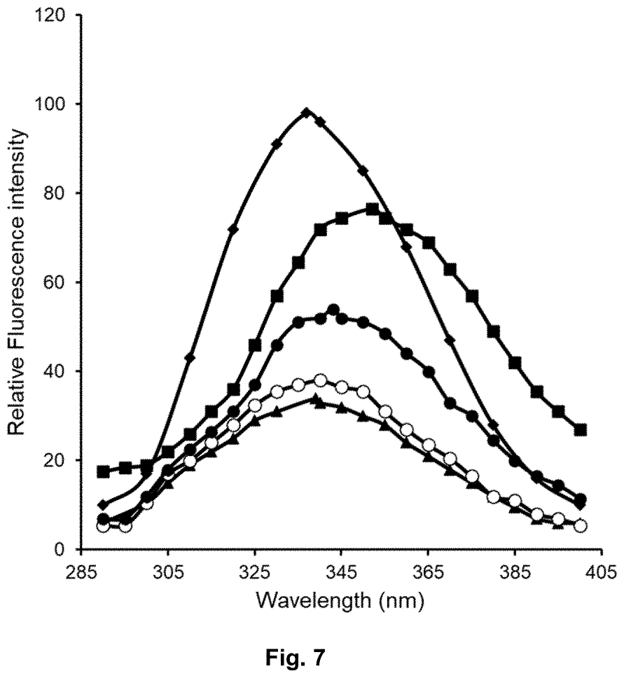

Conformational Changes in WPI-Dextran Conjugates: The fluorescence spectra of PPP conjugates of 6 kDa dextran at various extent of conjugation/phosphorylation is shown in FIG. 7. The fluorescence spectra of native WPI in 10 mM phosphate buffer and in 6 M urea are also shown in FIG. 7. At low extent of phosphorylation/conjugation, i.e., at WPI:POCl₃ ratio of 1:0.1 and 1:0.5, the fluorescence intensity was quenched without causing a major shift in λₘₐₓ of fluorescence, indicating that no major structural changes in the protein occurred at these levels of phosphorylation/conjugation. At WPI:POCl₃ ratio of 1:2 however, the fluorescence spectrum of the PPP conjugate red shifted, with the λₘₐₓ shifting from 337 nm for the native WPI to 345 nm for the PPP conjugate, indicating that the tertiary structure of the protein in the PPP conjugate was greatly altered at this extent modification. However, even at this high extent of modification, both the change in fluorescence intensity and the red shift in λₘₐₓ was lower for the PPP conjugate than those of WPI in 6M urea. This suggested that structural changes caused by phosphorylation/conjugation were not as extensive as in 6M urea solution, and the PPP conjugate retained some folded conformation despite an increase in net negative charge resulting from the attached phosphate groups. Similar results also were obtained with PPP conjugates of 10 kDa dextran (data not shown), strongly suggesting that the molecular weight of dextran played no significant role in the conformational change.

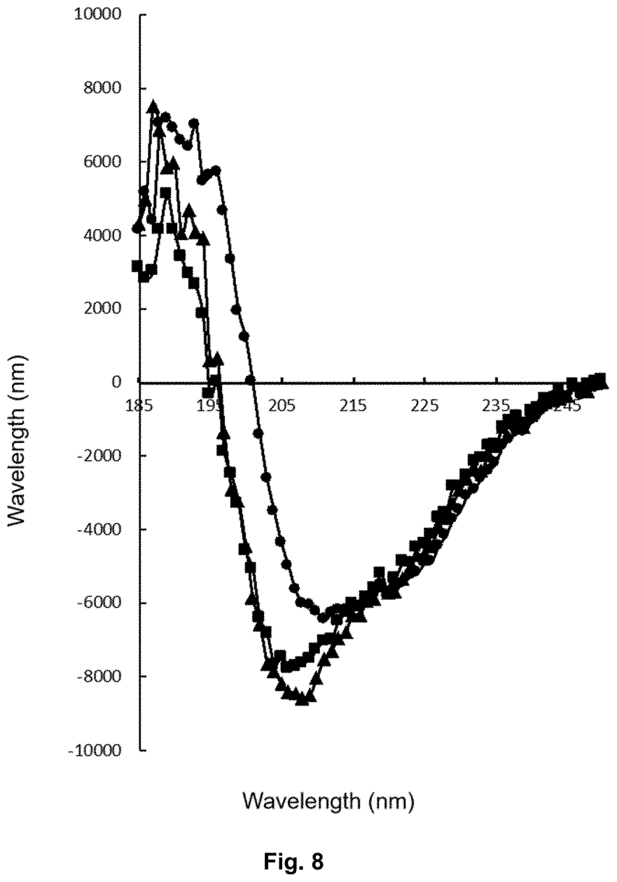

The changes in the secondary structure of the protein were studied using CD spectroscopy. The CD spectra of native WPI, phosphorylated WPI, and PPP conjugate are shown in FIG. 8. Secondary structure estimation using the method of Chang et al (1978) revealed that the native WPI contained 15% α-helix, 55% β-sheet, 5% β-turns, and 25% aperiodic structures, which were consistent with values reported in the literature (Damodaran, 1989). See Chang, C. T.; Wu, C. S. C.; Yang, J. T. Circular dichroic analysis of protein conformation: inclusion of the beta-turns. Anal. Biochem. 1978, 91, 13-31 and Damodaran, S. Influence of protein conformation on its adaptability under chaotropic conditions. Int. J. Biol. Macromol, 1989, 11, 2-8. The secondary structure content of the protein did not change significantly upon either phosphorylation or conjugation with dextran (Table 2). These result suggested that phosphorylation/conjugation altered only the tertiary structure, but not the secondary structure of the protein.

TABLE 2
Secondary structure estimates for native WPI,
phosphorylated WPI, and WPI-dextran conjugate.^{a}

| | α-helix | β-sheet | β-turns | Aperiodic |
|---|---|---|---|---|
| Sample | (%) | (%) | (%) | (%) |
| Native WPI | 15 | 55 | 5 | 25 |
| Phosphorylated WPI | 10 | 50 | 5 | 35 |
| PPP conjugate | 10 | 50 | 5 | 35 |

^{a}The WPI:POCl₃ ratio used in the preparation of the conjugate was 1:1.

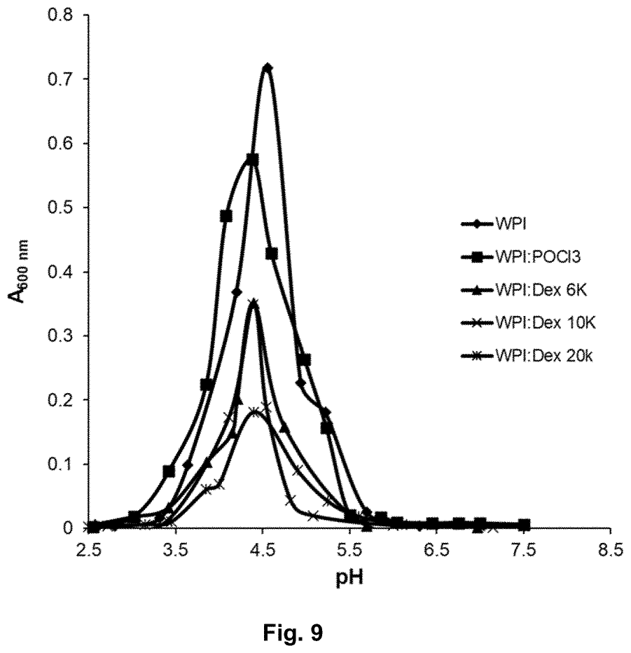

pH Solubility Profile of WPI-Dextran Conjugates: The pH solubility profile was studied for the native WPI, phosphorylated WPI, WPI-dextran conjugates (6 kDa, 10 kDa, and 20 kDa) between pH 2.5 and 7.5. Compared to the phosphorylated WPI and the WPI-dextran conjugates, the native WPI had a higher turbidity. i.e., decreased solubility at 600 nm. See FIG. 9. The isoelectric points of β-lactoglobulin and α-lactalbumin were evidenced at pH 5.2 and 4.56, respectively. However, upon phosphorylation, which increased the net negative charge of the WPI-dextran conjugate, the solubility increased and the isoelectric point shifted towards pH 4.38.

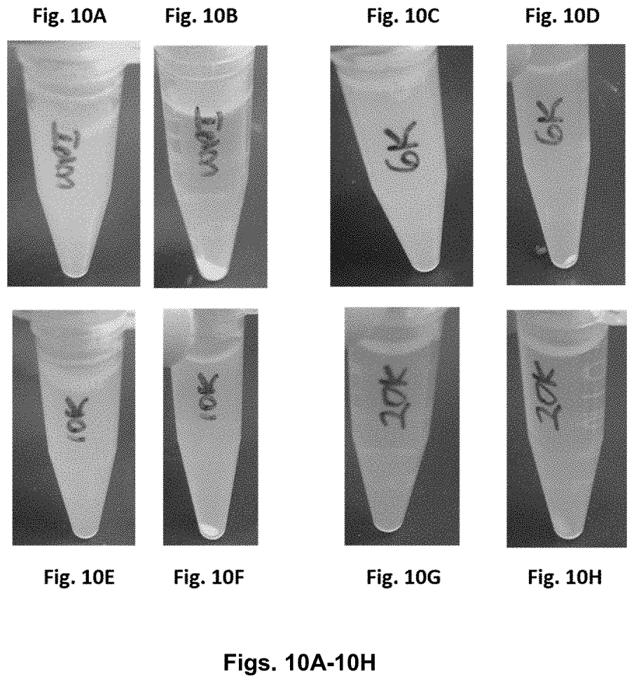

The visual appearances of the turbidity of control WPI and WPI-dextran conjugates at pH 4.56 before and after centrifugation at 11,000 g for 2 min are shown in FIGS. 10A through 10H. As shown in FIG. 10A, the turbidity was high for native WPI at pH 4.56, an upon centrifugation a large amount of the precipitate sedimented at the bottom of the tube (FIG. 10B). In contrast, the conjugates of dextran 6 kDa,10 kDa, and 20 kDa showed much less turbidity (see FIGS. 10C, 10E, and 10G, respectively) and remained stable as colloidal suspensions even after centrifugation (see FIGS. 10D, 10F, and 10H, respectively). The turbidity of the WPI-20 kDa dextran conjugate at pH 4.56 was much lower and the amount of sediment formed after centrifugation was minimal than the other conjugates, indicating that conjugation of WPI with 20 kDa dextran resulted in a more stable colloidal particle as compared to 6 kDa and 10 kDa dextrans.

Reacting a mixture of WPI and dextran with POCl₃ at pH 10 -10.5 under “molecular crowding” conditions resulted in the formation of protein-polysaccharide conjugates cross-linked via a phosphate bridge. The extent of conjugation increased with the weight ratio of protein to POCl₃ used in the reaction. This was confirmed by SDS-PAGE gels with Coomassie Blue G-250 staining for proteins and PAS reagent for staining carbohydrates. ³¹P NMR of the PPP conjugate also confirmed the presence of —N^{pro}—PO₂⁻—O^{Dex} cross-link in the PPP conjugate. Quantitative analysis of the ³¹P NMR revealed that about 32% of the phosphorylated lysine residues in the PPP conjugate were involved in the —N^{pro}—PO₂⁻—O^{Dex} bond. Incubation of PPP conjugate at pH 1.5 at 40° C. caused hydrolysis of all N-phosphates, including the —N^{pro}—PO₂⁻—O^{Dex} cross-link, and release of the amine groups in a time-dependent manner. This indicated that lysine residues cross-linked to the polysaccharide in the PPP conjugate could become biologically available during transit through the gastro-intestinal tract.

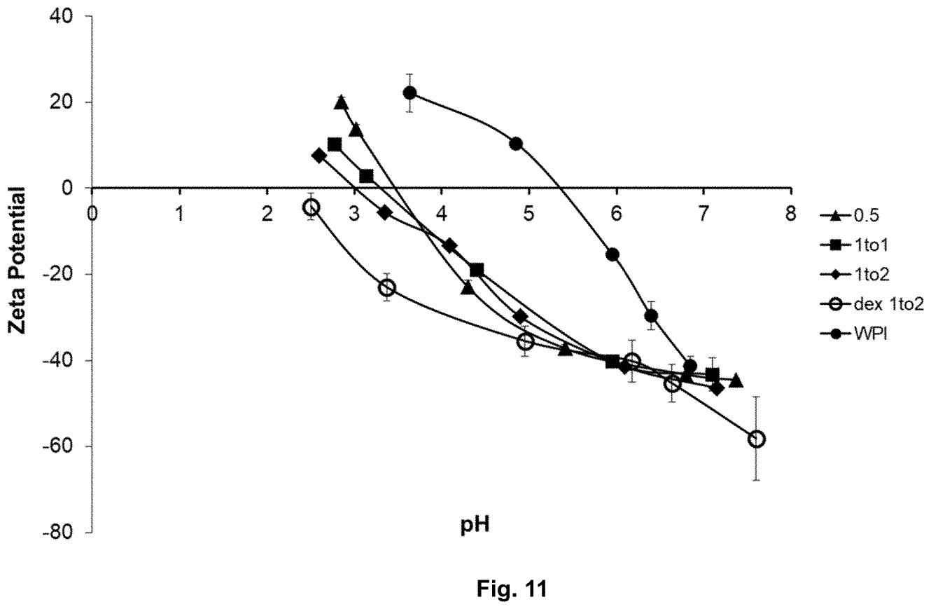

Zeta potential as a function of pH for various WPI-dextran (6 kDa) conjugates at various extents of phosphorylation were determined. The results are shown in FIG. 11. A larger absolute value for zeta potential correlates with the ability of the conjugate to form a stable colloid. That is, conjugates having a zeta potential close to zero will tend to coagulate or flocculate in solution. The larger the absolute value of zeta potential (positive or negative), the more stable will be colloids formed from the conjugate.

It should be noted that di-starch phosphate, which is widely used as modified starch in various food products (e.g., bread and bakery products, breakfast cereals, pastas, and snacks), is phosphate cross-linked amylose. In a recent ‘scientific opinion’ report, the European Food Safety Authority (2010) has declared that phosphated di-starch phosphate is safe for human consumption. This likely is also true of protein-phosphate-polysaccharide (PPP) conjugate as well. Furthermore, post-translational phosphorylation of basic amino acid residues (lysine, histidine, and arginine) in proteins is very common in many biologically important proteins (Ciesla, 2010). Thus, the PPP conjugate disclosed herein is likely biologically safe for human consumption.

What is claimed is:

1. A compound comprising a protein moiety covalently linked to a polysaccharide moiety via a phosphate bridge.

2. The compound of claim 1, wherein the polysaccharide moiety comprises a saccharide residue selected from the group consisting of ribose, arabinose, xylose, lyxose, allose, altrose, glucose, mannose, gulose, idose, galactose, talose, ribulose, xylulose, psicose, fructose, sorbose and tagatose.

3. The compound of claim 1, wherein the phosphate bridge is bound to an oxygen atom on the protein moiety.

4. The compound of claim 1, wherein the phosphate bridge is bound to a nitrogen atom on the protein moiety.

5. The compound of claim 1, wherein the polysaccharide moiety is dextran.

6. The compound of claim 1, wherein the protein moiety comprises a whey protein.

7. The compound of claim 6, wherein the polysaccharide moiety comprises a saccharide residue selected from the group consisting of ribose, arabinose, xylose, lyxose, allose, altrose, glucose, mannose, gulose, idose, galactose, talose, ribulose, xylulose, psicose, fructose, sorbose and tagatose.

8. The compound of claim 6, wherein the phosphate bridge is bound to an oxygen atom on the protein moiety.

9. The compound of claim 6, wherein the phosphate bridge is bound to a nitrogen atom on the protein moiety.

10. The compound of claim 6, wherein the polysaccharide moiety is dextran.

11. A method of making a conjugate having a protein moiety and a saccharide moiety linked by a phosphate bridge, the method comprising contacting, in a solvent, a protein, a polysaccharide, and POCl₃ for a time, at a temperature, and at a pH wherein a reaction occurs that yields a protein-phosphate bridge-polysaccharide (“PPP”) conjugate.

12. The method of claim 11, wherein the solvent is water.

13. The method of claim 11, wherein the pH is selected from a range of from about pH 8 to about pH 14, or from about pH 8 to about pH 12, or from about pH 9 to about pH 11, or from about pH 10 to about pH 11.

14. The method of claim 11, wherein the protein is present in a concentration of from about 5% (w/v) to about 50% (w/v) with respect to the solvent, and the polysaccharide is present in a concentration of from about 5% (w/v) to about 50% (w/v) with respect to the solvent.

15. The method of claim 11, wherein POCl₃ is present at a protein-to-POCl₃ ratio (w/w) of from about 0.5 to about 5.

16. The method of claim 11, wherein the temperature is from about 10° C. to about 30° C.
